(19) United States
(12) Patent Application Publication
(10) Pub. No.: US 2009/0062187 A1
(43) Pub. Date: Mar. 5, 2009

(54) Use of Hmgb1 for Wound Healing

(76) Inventors: Marco Bianchi, Milano (IT); Maurizio Colognesi Capogrossi, Rome (IT); Antonia Germani, Rome (IT)

Correspondence Address: JUNEAU PARTNERS, P.O. BOX 2516, ALEXANDRIA, VA, 22301, US

(21) Appl. No.: 11/658,299

(22) PCT Filed: Jul. 20, 2005
(86) PCT No.: PCT/IT2005/000422

§ 371 Date: Nov. 14, 2008

Related U.S. Application Data
(60) Provisional application No. 60/589,455, filed on Jul. 20, 2004.

(51) Int. Cl. A61K 38/16; A61P 17/02; A61P 3/10; A61P 29/00

(52) U.S. Cl. 514/12

(57) ABSTRACT

The present invention describes the role of HMGB1 in improving wound healing, in particular in a model of diabetes. Other evidences based on the effect of glycyrrhizin, the underexpression of HMGB1 in the skin and fibroblast of diabetic subjects, the accumulation of RAGE in the skin of diabetic mice and the chemoattractant properties of HMGB1 on normal and diabetic human cells demonstrate that HMGB1 can be advantageously utilized for preparing medicament specifically devoted to wound healing, in particular for diabetic subjects.

Chronic ulcers and defective tissue repair represent a major health problem. Conventional therapeutic approaches are not sufficient to guarantee an adequate healing in chronic ulcers and recurrence is frequent. Wound healing involves the coordinated interaction of numerous cell types and is characterized by three phases: inflammation, proliferation and remodelling (1). These events are stimulated by a number of growth factors and cytokines including Fibroblast growth factor family (FGFs), Transforming growth factor alpha (TGFalpha), Epidermal growth factor (EGF), Platelet derived growth factor BB (PDGF BB), Interleukin 8 (IL-8), Monocyte chemo-attractant protein 1 (MCP-1) (2).

In diabetes, abundant evidences exist that the phases of wound healing are deregulated, and decreased chemotaxis of inflammatory cells into the wound leads to diminished availability of growth factors important for effective wound repair. In addition, excess protease activity and increased microbial load together with the eventual co-existence of peripheral vascular disease impede wound healing in diabetic patients (3-5).

HMGB1 is a new cytokine released from monocyte-macrophage in response to proinflammatory cytokines and from necrotic cells (6-8). Extracellular HMGB1 elicits proinflammatory responses in endothelial cells, by increasing the expression of vascular adhesion molecules as well as secretion of cytokines (TNFalpha) and chemokines (IL8 and MCP-1) (9). Several lines of evidences have demonstrated that the effects of HMGB1 are mediated by its binding to the receptor for advanced glycation products (RAGE), a multiligand receptor of the immunoglobulin superfamily. Recently it has been demonstrated that HMGB1 and its receptor RAGE induce migration and proliferation of smooth muscle cells and vessels associated stem cells (mesoangioblasts) (10, 11). WO2004/004763 discloses the use of HMGB1 in the treatment of tissue damage, namely cardiac and skeletal muscle. However this application does not provide any evidence on the advantageous use of HMGB1 in diabetic subjects, wherein wound repair is of critical relevance. In addition, there is no suggestion in the prior art to test HMGB1 for wound healing, given the inflammatory activity of the molecule.

In the present invention, the authors have found that i) HMGB1 improves wound healing, in particular in an animal model of diabetes, ii) The HMGB1 inhibitor, Glycyrrhizin, impairs wound healing in normal mice, iii) HMGB1 is underexpressed in the skin of diabetic mice and fibroblasts of diabetic patients, iv) HMGB1 receptor, RAGE accumulates in the skin of diabetic mice and v) HMGB1 has a chemoattractant effect on human normal and diabetic fibroblasts and keratinocytes. Therefore the molecule can be advantageously utilized for preparing medicament specifically devoted to wound healing, in particular for diabetic subjects.

It is then an object of the instant invention the use of HMGB1 or of pharmacologically active analogues or derivatives thereof for the preparation of a medicament for wound healing.

In the present invention, wound healing comprises ulcers, venous ulcers, pressure ulcers, burns healing, and any other wound care treatment.

The composition of the invention shall be prepared by selecting appropriate concentration, administration and dosage form. Preferred administration forms include oils, ointments, spray foams, creams, also on a solid support as a medicated patch for topical use. Proper diluents, emollients, adjuvants, excipients and, optionally, other pharmacologically active compounds to get a multi drug composition are utilized. A preferred pharmacologically active compound is an anti-inflammation agent. The composition of the invention is also usable in the cosmetic field for the preparation of regenerative products, as for example anti-aging creams or sera.

The invention will be now described by means of non limiting examples, making reference to the following figures:

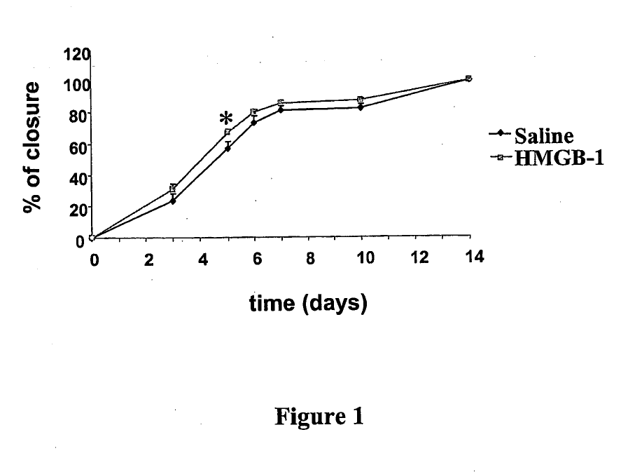

FIG. 1: Effect of HMGB1 on wound healing in normal CD1 mice. Mice received directly in the wound area a saline solution or 20 μl of a solution containing 200 ng of HMGB1. The percentage of wound closure was calculated at day 0, 3, 5, 6, 7, 10, 14 after wounding as indicated in the method section. Data are mean±SEM, n=10 mice per group. The percent of wound closure of mice treated with HMGB1 (200 ng) was significantly higher than the one in mice treated with saline at day 5. * p<0.03 vs. saline treated group. Statistical significance between two measurements was evaluated by unpaired Student's t test.

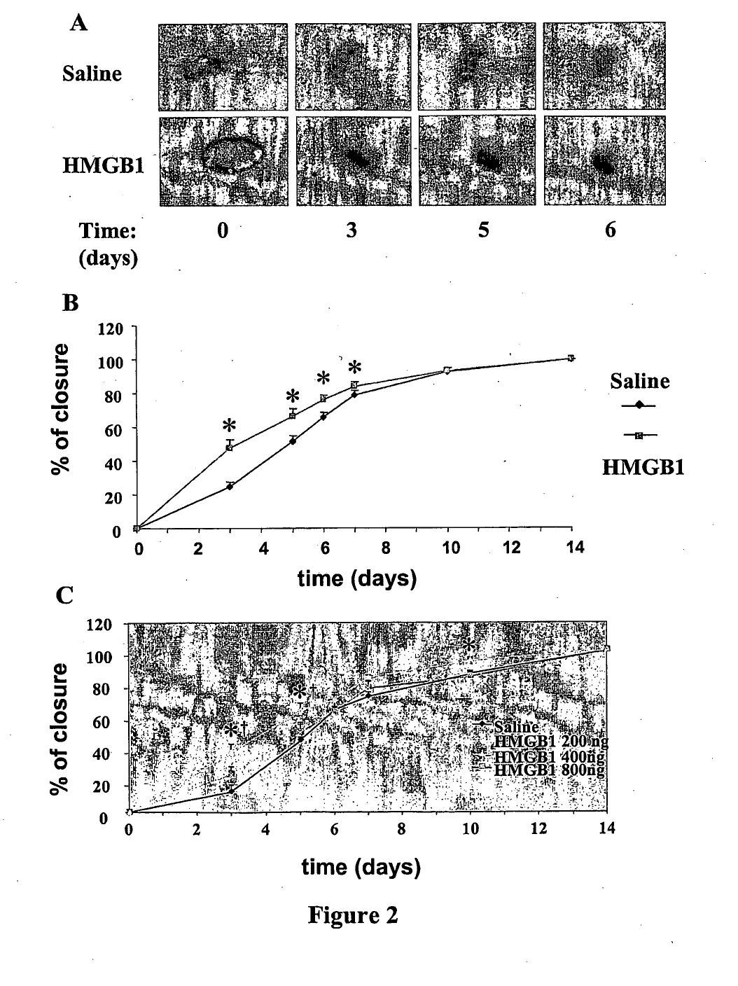

FIG. 2: Effect of HMGB1 on wound healing in diabetic CD1 mice. Mice were rendered diabetic by intraperitoneal injection of streptozotocin (1.2 mg/mouse/day) for 5 consecutive days. Mice received directly in the wound area a saline or a HMGB1 (200 ng) solution. (A) Wound photograph were taken from day 0 (immediately after wounding) to day 6 after wounding. (B) The percentage of wound closure was calculated at day 0, 3, 5, 6, 7, 10, 14 after wounding as indicated in the method section. Data are mean±SEM, n=6 mice per group. The healing rate of diabetic mice treated with HMGB1 (200 ng) was significantly higher than the one in diabetic mice treated with saline. * p<0.03 vs. saline treated group. Statistical significance between two measurements was evaluated by unpaired Student's t test. (C) Effect of increasing concentrations of HMGB1 (200, 400 and 800 ng) on wound closure in diabetic mice. The percentage of wound closure was calculated at day 0, 3, 5, 6, 7, 10, 14 after wounding as indicated in the method section. Data are mean±SEM, n=10 mice per group. * p<0.05 and † p<0.05 vs saline solution and HMGB1 200 ng, respectively.

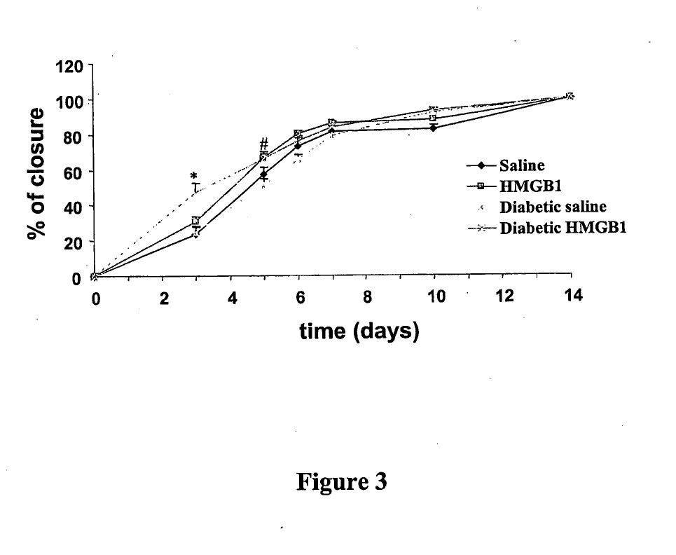

FIG. 3: Comparison of HMGB1 effect on wound healing in normal and diabetic CD1 mice. Mice were rendered diabetic by intraperitoneal injection of streptozotocin (1.2 mg/mouse/day) for 5 consecutive days. Animals received directly in the wound area a saline or a HMGB1 (200 ng) solution. The percentage of wound closure was calculated at day 0, 3, 5, 6, 7, 10, 14 after wounding as indicated in the method section. Data are mean±SEM, n=10 mice per group. * p<0.01 vs all treatments; #HMGB1 and diabetic HMGB1 vs saline p<0.05.

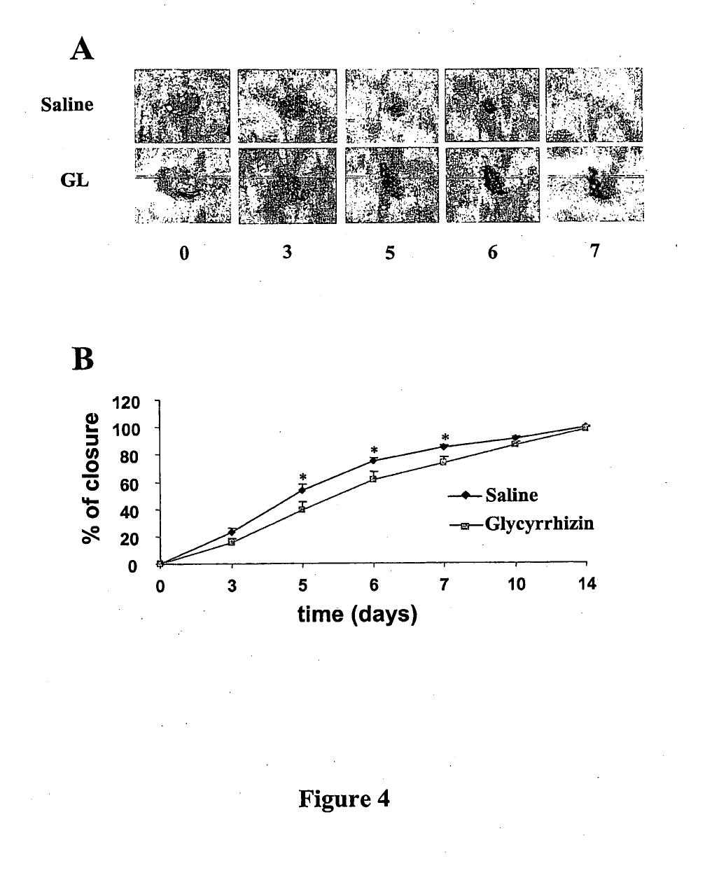

FIG. 4: Effect of glycyrrhizin on wound healing in normal CD1 mice. Mice received directly in the wound area a saline or a glycyrrhizin (250 kg/mouse in 30 μl of PBS) solution. (A) Wound photographs were taken from day 0 (immediately after wounding) to day 7 after wounding. (B) The percentage of wound closure was calculated at day 0, 3, 5, 6, 7, 10 and 14 after wounding as indicated in the method section. Data are mean±SEM, n=10 mice per group. The healing rate of mice treated with glycyrrhizin was significantly lower than the one in mice treated with saline. * p<0.05 vs. glycyrrhizin treated group. Statistical significance was evaluated by unpaired Student's t test.

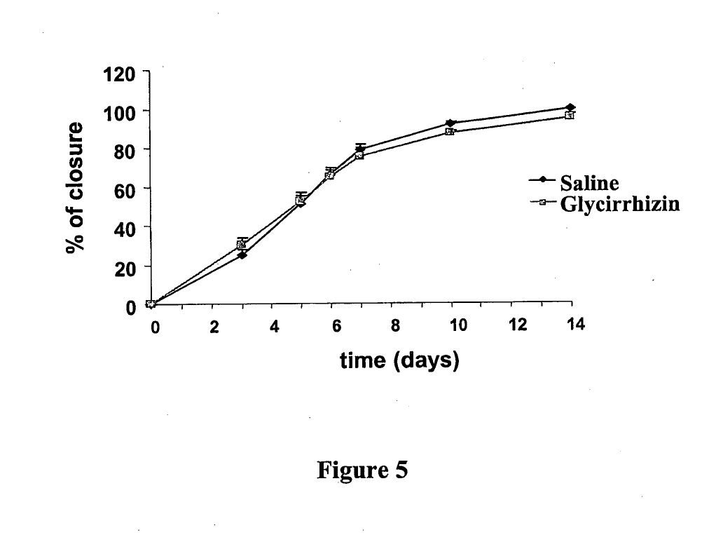

FIG. 5: Effect of glycyrrhizin on wound healing in diabetic CD1 mice. Mice were rendered diabetic by intraperitoneal injection of streptozotocin (1.2 mg/mouse/day) for 5 consecutive days. Mice received directly in the wound area a saline or a glycyrrhizin (250 μg/mouse in 30 μl of PBS) solution. The percentage of wound closure was calculated at day 0, 3, 5, 6, 7, 10 and 14 after wounding as indicated in the method section. Data are mean±SEM, n=9 mice per group. No significant differences between the two groups were observed.

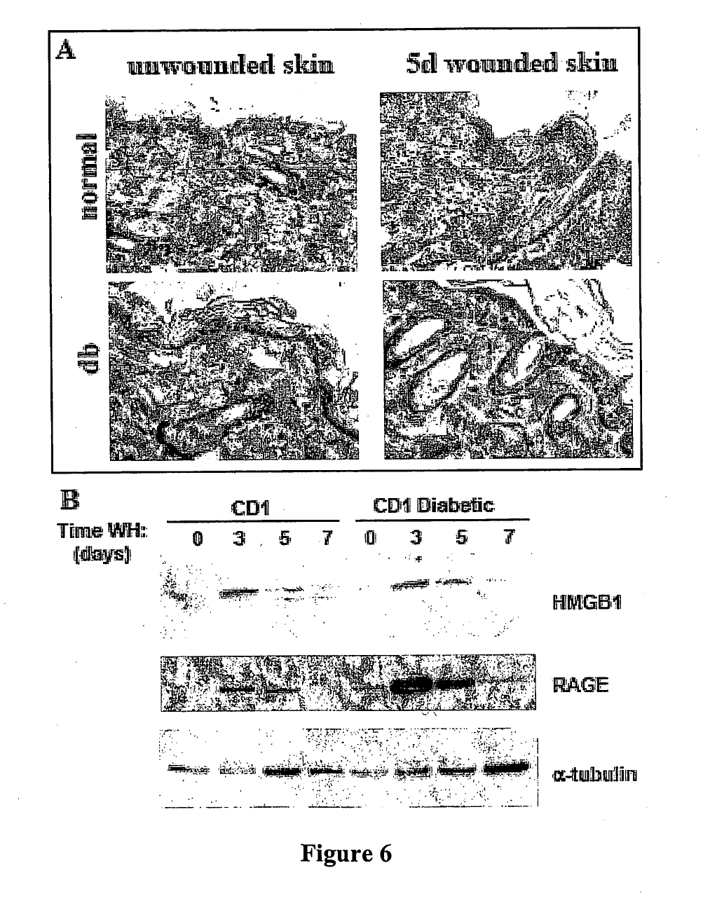

FIG. 6: Localization and expression of HMGB1 in wounded skin of normal and diabetic CD1 mice. Mice were rendered diabetic by intraperitoneal injection of streptozotocin (1.2 mg/mouse/day) for 5 consecutive days. (A) HMGB1 immunohistochemical analysis in normal and diabetic (db) mouse skin before (left panel) and 5 days after wound (right panel). (B) HMGB-1 and RAGE western blot analysis in normal and diabetic mice skin at 0, 3, 5 and 7 days after wound. The same filter was probed with anti alpha-tubulin Mab to normalize protein concentration.

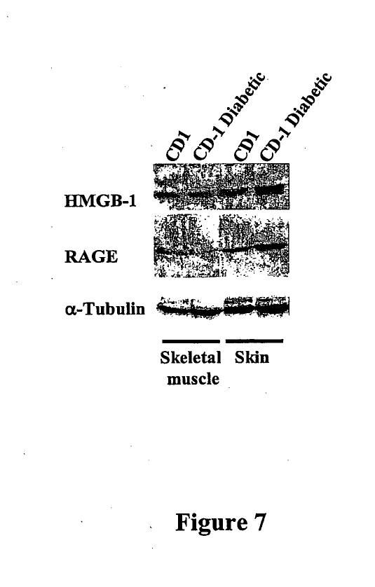

FIG. 7: HMGB1 and RAGE western blot analysis in skeletal muscle and skin of 2 months old normal and diabetic CD1 mice. Mice were rendered diabetic by intraperitoneal injection of streptozotocin (1.2 mg/mouse/day) for 5 consecutive days. The same filter was probed with anti alpha-tubulin Mab to normalize protein concentration.

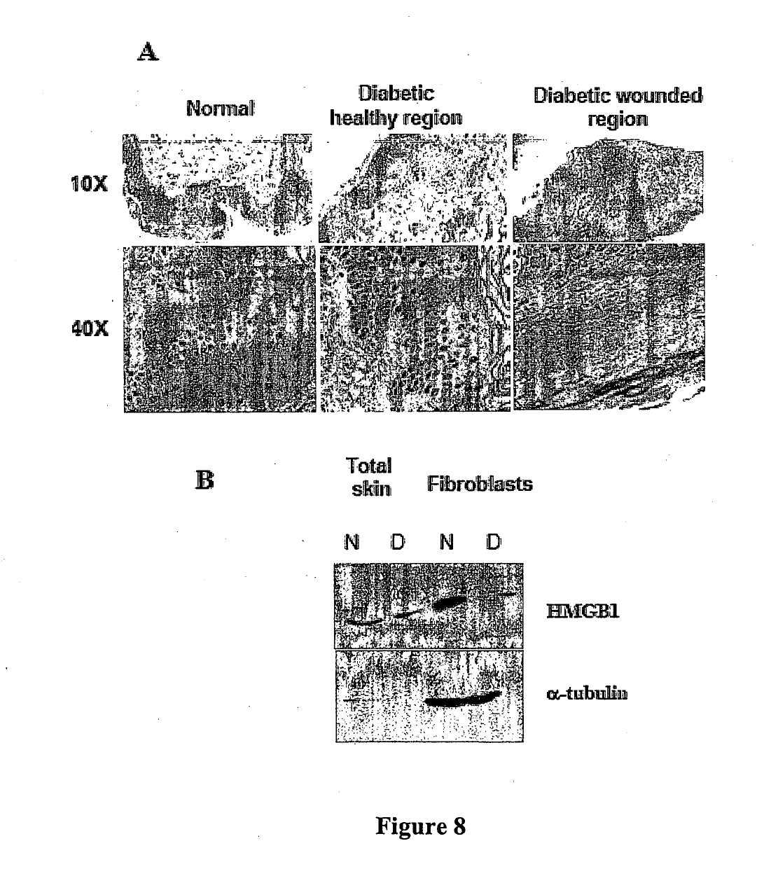

FIG. 8: Localization and expression of HMGB1 in normal and wounded skin of diabetic patients. (A) HMGB1 immunohistochemical analysis in normal human skin (Left panel), in diabetic skin (middle panel) and in diabetic ulcer skin (right panel). (B) HMGB1 western blot analysis in normal (N) and diabetic (D) human skin and fibroblasts. The same filter was probed with anti alpha-tubulin Mab to normalize protein concentration.

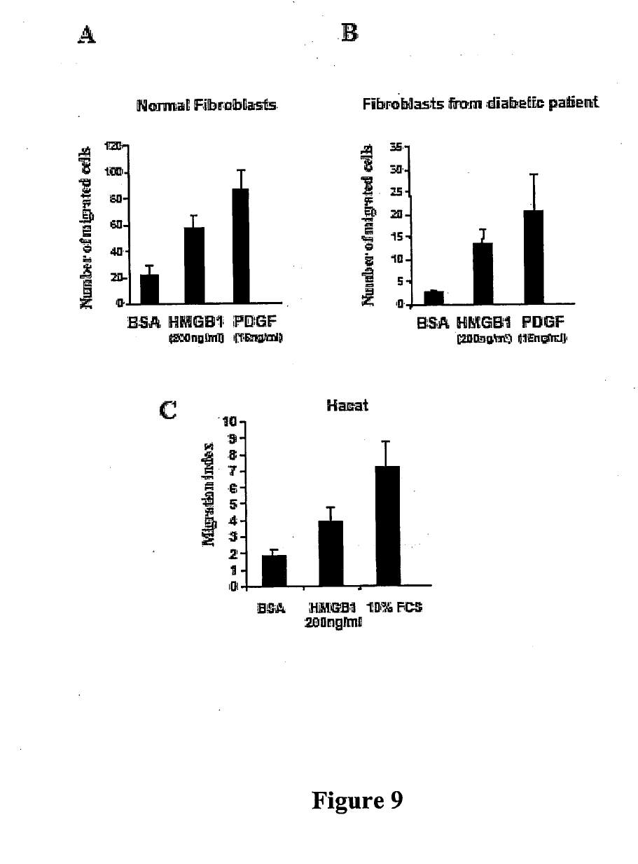

FIG. 9: Effect of HMGB1 on human fibroblasts and keratinocytes migration. Normal (A) and diabetic (B) fibroblasts were obtained from human biopsies. 0.4×10⁶ cells/ml were placed in upper compartment of the modified Boyden chambers. HMGB1 (200 ng/ml) or PDGF (15 ng/ml) was added to the lower compartment and incubated for 6 hrs at 37° C. (C) Hacat cells were obtained from ATCC and migration experiments performed as described in (A-B). After staining with Giemsa solution, migrated cells were quantified by counting nuclei in five random microscope fields (40×). The data are expressed as migration index (fold increase in number of migrated cells relative to number of migrated cells in the absence of HMGB1 or PDGF) and are mean±SD of at least 4 independent experiments performed in triplicate. Statistical significance was evaluated by unpaired Student's t test.

### MATERIALS AND METHODS

### Animal Wound Model

CD1 male mice were obtained from Charles River (Calco, LC, Italy). Mice were rendered diabetic by intraperitoneal injection of streptozotocin (Sigma-Aldrich, St Louis, Mo., USA) at 1.2 mg/30 g weight/day for 5 consecutive days. After 7 days, glycemia was measured and animals with glycemia of 200 to 400 mg/dl were selected for further studies. Mice were anesthetized with intraperitoneal injection of 2.5% Avertin (100% Avertin: 10 g of 2,2,2-tribromoethyl alcohol and 10 ml of tert-amyl alcohol, Sigma). Their dorsum was clipped free of hair and a full thickness wound of 3.5 mm diameter was created using a biopsy punch.

### Drug Treatment

HMGB1 treatment was performed directly in the wound area by injecting 200 ng, 400 ng or 800 ng of purified protein (13) in 20 μl of saline solution at time immediately after wound. Control groups received 20 μl of saline solution in the wound. Glycyrrhyzin, GL (12) was administered topically in the wound area every two days from day 0 to day 14 after wound creation. The concentration used for each administration was 250 μg/mouse in 30 μl of PBS. Control mice received vehicle (PBS).

### Determination of Wound Closure Rate in Mice

Animals were photographed at day 0, 3, 5, 6, 7, 10, and 14 after wound. Pictures were digitally processed and areas of wounds were calculated using the KS300 system (Zeiss, Jena GmbH, Germany). For each sample the percentage of wound closure was calculated as the ratio:

$$( 1 - \frac{Wound area at each time point}{Wound area at time 0} ) \times 100$$

Where time 0 is the time immediately after the wound. The groups included 6 to 10 animals. Results are presented as mean±standard error. Statistical significance between two measurements was evaluated by unpaired Student's t test.

### Immunohistochemical Analysis of Mice and Human Skin

Sections (3 μm thickness) obtained from human skin biopsies and mouse skin tissues were deparaffinized, after short treatment in microwawe, rinsed with PBS, incubated at room temperature for 20 minutes with a solution of methanol containing 0.03% H2O2 and were blocked for 1 hr with 10% rabbit or goat serum in 5% BSA and incubated overnight at 4° C. with rabbit polyclonal anti-HMGB1 antibody (1 μg/ml BD Pharmingen). HMGB1 detection was performed with biotinylated secondary antibodies (7.5 μg/ml, Vector Laboratories) and incubated avidin-biotinylated peroxidase complex (ABC Elite Kit, Vector Laboratories). The stain was visualized by treatment for 10 minutes in a 0.05% solution of 3-diaminobenzidine (DAB) and 0.01% H₂O₂ in 0.1 M PBS. Sections were counterstained with hematoxylin to identify nuclei.

### Western Blot Analysis of Mice and Human Samples

Excised wounds, adductor skeletal muscle or cultured fibroblasts were lysed in RIPA buffer containing 10 mM Tris-HCl (pH 7.4), 150 mM NaCl, 1% NP40, 1% deoxycolic acid, 0.1% SDS, 10% Glycerol and protease inhibitors. Equal amounts of total cellular proteins (100 μg/lane) were resolved by 10% SDS-polyacrylamide gel electrophoresis and transferred to nitrocellulose membrane (Amersham Pharmacia Biotech, Little Chalfont, UK). Membranes were probed with specific antibodies (1 μg/ml anti HMGB1; 0.4 μg/ml anti RAGE and 0.1 μg/ml anti alpha-tubulin MAb) followed by horseradish peroxidase-coupled secondary antibodies and developed by a chemiluminescence-based detection system (FCL, Amersham Pharmacia Biotech).

### Patient Biopsies

After Local Ethical Committee approval and signed informed consent, biopsies were obtained from both normal and type II diabetic patients undergoing to routine surgery from the edge of surgical incision under local anesthesia (n=4). Similarly biopsies were also obtained from the edge of foot ulcer lesions in diabetic patients (n=2).

### Human Fibroblast Isolation and Culture

For fibroblasts isolation, biopsies were seeded on 6 mm diameter tissue culture dish containing 10% fetal bovine serum (FBS, Euroclone Inc., Milan, Italy), 20 mM Glutamine, 100 U/ml Penicillin and 100 mg/ml Streptomycin (Gibco BRL, Paisley, UK). Fibroblasts began to grow out from the explants after 7-10 days, became confluent within 3-5 weeks and were used at passage 2-3 for the chemotaxis assay.

### Keratinocyte Culture

Hacat human cells were obtained according to (14).

### Chemotaxis Assays

Chemotaxis was performed in 48-microwell chemotaxis chambers (Neuroprobe, Cabin John, Md.) using 8 μm pore-size polycarbonate filters (Costar Scientific Corporation, Cambridge, Mass., USA) coated with murine collagen type IV (Becton-Dickinson, Bedford, Mass., USA).

The lower compartment of each chamber was filled with 28 μl DMEM with 0.1% BSA. HMGB1 was added at the concentration of 200 ng/ml, PDGF (15 ng/ml) and BSA were used as negative and positive controls for migration respectively. Each well of the upper compartment was filled with 50 μl DMEM with 0.1% BSA, containing either human fibroblasts or keratinocytes (0.4×10⁶ cells/ml). Each point was run in triplicate. After 4 hr incubation at 37° C. in a 5% CO₂ humidified atmosphere, the chemotaxis assay was stopped, cells on the filter were fixed and stained using Diff Quik (Dade AG, Dudingen, Switzerland). Cells on five random fields on the lower face of the filter were counted at 40× magnification and migration index was calculated by dividing the number of migrated cells in the presence of chemoattractants by the cells migrated in response to DMEM with 0.1% BSA.

### Results

### Effect of HMGB1 on Wound Healing in Normal and Diabetic Mice

To test the role of HMGB1 on wound healing, full-thickness excised wound was created on the back of normal and diabetic CD1 mice. HMGB1 was injected in the peripheral wound area immediately after wounding. Analysis of wound area was performed through digital processing of pictures (representative examples are shown FIG. 2A) taken at different time points after the wound. The rate of wound healing is expressed as percentage of closure. As seen in FIG. 1, HMGB1 treatment increased wound closure in normal CD1 mice. The difference between untreated and HMGB1-treated CD1 mice was significant 5 days after the treatment (p<0.03). A trend towards increased wound closure in HMGB1-treated group was observed at all time points evaluated from day 3 to day 10, indicating an improvement in wound closure in HMGB1-treated mice (FIG. 1). Similarly, 3 days after wounding, HMGB1 treatment significantly increased wound closure in diabetic mice when compared to control, saline treated mice: 24.6±14% in control mice and 47.5±15.8% in HMGB1-treated mice (n=10, p<0.03). This effect persisted at day 5 (66.5±13% vs 51±16.7%; p<0.02), day 6 (72.2±9% vs 65.9±12.2% p<0.03) (FIG. 2A, B). Complete wound closure was evident by day 14 in both groups.

The authors found that 3 days after wounding, higher dose (400 ng in 20 ul) of HMGB1 induced a larger wound closure compared to a lower dose (200 ng) (FIG. 2C). HMGB1 administered at 800 ng produced the same effect than the dose of 200 ng (FIG. 1C). Interestingly, when normal and diabetic mice were compared, the authors found that diabetic HMGB1-treated mice healed better than normal HMGB1-treated CD1 mice (FIG. 3). At day 3 after wounding the percentage of wound closure was 47.5±15.8% in HMGB1-treated diabetic mice vs 30.5±8.5% (p<0.01) in HMGB1-treated normal mice, and 23.5±11.2% (p<0.003) in saline treated normal mice (FIG. 3).

### Effect of HMGB1 Inhibitor, Glycyrrhizin (GL) on Wound Closure in Normal and Diabetic Mice

The effect of the GL (12) on wound closure was tested in normal and diabetic mice. GL was administered topically in the full excised wound of mice, every two days from day 0 to day 14 after wound creation. Control mice received vehicle (PBS) (FIG. 4 A,B). The authors observed that administration of GL slowed down wound closure (FIG. 4 A,B). At day 3 wound areas were reduced by 23±2% and 15±3.5% of the initial size of the wound, in PBS and GL treated mice, respectively. The difference in wound closure between control and GL treated mice became statistically significant with a wounded area reduced by 55.2±3% vs. 38.7±6% at day 5, by 74.2±2.4% vs, 61.2±5.9% at day 6, and 83.24±1.7% vs. 73.3±4.5% at day 7 in control vs. GL treated group, respectively (FIG. 4).

As reported in FIG. 5, glycyrrhizin (250 μg in one dose in the wound area immediately after wound) did not significantly modify wound closure in diabetic mice suggesting that HMGB1 is not spontaneously released in the wound of diabetic mice.

### Localization of HMGB1 in the Skin of Normal and Diabetic Mice

Immunohistochemical analysis was performed on sections obtained from full-thickness excised wound created on the back of diabetic CD1 mice. HMGB1 was detected in the nucleus of dermal and epidermal cells from normal and diabetic mice skin. At day 5 after wound HMGB1 localized in the cytoplasm of all cell types in both normal and diabetic skin (FIG. 6A).

### Expression of HMGB1 and its Receptor (RAGE) in the Skin of Normal and Diabetic Mice

Western blot analysis did not reveal a significant difference in HMGB1 levels between normal and diabetic CD1 mouse skin at day 0. By contrast, from day 3 to day 7 after skin punching, lower levels of HMGB1 were detected in skin obtained from diabetic mice compared to skin from normal mice (FIG. 6B). RAGE expression was higher in diabetic skin compared to normal skin at day 0 and strongly accumulated in diabetic skin mice at day 3 and 5 after wound. (FIG. 6B). Interestingly, RAGE accumulation did not occur in skeletal muscle tissue of diabetic mice (FIG. 7).

### Localization and Expression of HMGB1 in Wounded Skin of Normal and Diabetic Patients

HMGB1 distribution was analyzed in skin biopsies obtained from normal and diabetic patients. Similar to what observed with normal and diabetic mice skin, immunohistochemical analysis revealed that HMGB1 distribution was similar between normal and diabetic human skin and is restricted to the nucleus of both epidermal and dermal cells (FIG. 8A). To address whether HMGB1 distribution was modified in chronic nonhealing wounds, skin biopsies were taken from the edge of the ulcers of human diabetic patients. In nonhealing ulcers HMGB1 localized in the cytoplasm of both epidermal and dermal cells (FIG. 8A).

HMGB1 contents of total cellular skin extract and fibroblasts obtained from normal and diabetic human skin were then evaluated by western blot analysis. Similar levels of HMGB1 were detected in human normal and diabetic total cellular skin extract. However, HMGB1 content was significantly reduced in human diabetic fibroblasts (FIG. 8B).

### Effect of HMGB1 on Human Fibroblast and Keratinocyte Migration

Rapid induction of keratinocyte and fibroblast migration into wounds is necessary for tissue repair. The authors examined whether human keratinocyte and fibroblast migration was modulated in response to HMGB1 in a multiwell chemotaxis chamber. Under the experimental conditions of the present study, HMGB1 exhibited a chemotactic effect at the concentration of 200 ng/ml in normal and diabetic fibroblasts (FIG. 9A,B) as well as on keratinocytes (HaCat cell line, FIG. 9C).

### BIBLIOGRAPHY

1. Martin, P. (1997) Science 276, 75-81.
2. Gillitzer, R. & Goebeler, M. (2001) J Leukoc Biol 69, 513-21.
3. Doxey, D. L., Ng, M. C., Dill, R. E. & Iacopino, A. M. (1995) Life Sci 57, 1111-23.
4. Shaw, J. E. & Boulton, A. J. (1997) Diabetes 46 Suppl 2, S58-61.
5. Wemer, S., Breeden, M., Hubner, G., Greenhalgh, D. G. & Longaker, M. T. (1994) J Invest Dermatol 103, 469-73.
6. Andersson, U., Wang, H., Palmblad, K., Aveberger, A. C., Bloom, O., Erlandsson-Harris, H., Janson, A., Kokkola, R., Zhang, M., Yang, H. & Tracey, K. J. (2000) J Exp Med 192, 565-70.
7. Scaffidi, P., Misteli, T. & Bianchi, M. E. (2002) Nature 418, 191-5.
8. Wang, H., Vishnubhakat, J. M., Bloom, O., Zhang, M., Ombrellino, M., Sama, A. & Tracey, K. J. (1999) Surgery 126, 389-92.
9. Fiuza, C., Bustin, M., Talwar, S., Tropea, M., Gerstenberger, E., Shelhamer, J. H. & Suffredini, A. F. (2003) Blood 101, 2652-60.
10. Degryse, B., Bonaldi, T., Scaffidi, P., Muller, S., Resnati, M., Sanvito, F., Arrigoni, G. & Bianchi, M. E. (2001) J Cell Biol 152, 1197-206.
11. Palumbo, R., Sampaolesi, M., De Marchis, F., Tonlorenzi, R., Colombetti, S., Mondino, A., Cossu, G. & Bianchi, M. E. (2004) J Cell Biol 164, 441-9. Epub 2004 Jan. 26.
12. Sakamoto, R., Okano, M., Takena, H. & Ohtsuki, K. (2001) Biol Pharm Bull 24, 906-11.
13. Muller S, Bianchi M E, Knapp S. Thermodynamics of HMGB1 interaction with duplex DNA. Biochemistry. 2001; 40:10254-61.
14. Boukamp P, Petrussevska R T, Breitkreutz D, Hornung J, Markham A & Fusenig N E. J. Cell Biol. 1988; 106:761-771.

1. A process of preparing a medicament for wound healing, comprising combining of HMGBI or of pharmacologically active analogues or derivatives thereof with one or more diluents and/or embodiments and/or adjuvants and/or excipients.

2. A method of treating a diabetic patient, comprising: administering HMGB1 or of pharmacologically active analogues or derivatives thereof to said patient in a manner sufficient for wound healing of diabetic subjects.

3. A pharmaceutical composition for use according to claim 1 comprising a wound healing effective but not inflammation inducing amount of HMGB1 or of active analogues or derivatives thereof, and diluents and/or emollients and/or adjuvants and/or excipients.

4. The pharmaceutical composition according to claim 3 for topical use.

5. The pharmaceutical composition according to claim 3 further comprising another pharmacologically active compound.

6. The pharmaceutical composition according to claim 5 wherein the pharmacologically active compound is an anti-inflammatory compound.

7. A medicated patch essentially comprising a solid support and the pharmaceutical composition according to claim 3.

8. A process for preparing a cosmetic product, comprising: combining HMGB1 or of pharmacologically active analogues or derivatives and a cosmetic carrier for the preparation of a cosmetic product.

9. A cosmetic formulation comprising a cosmetic effective but not inflammation inducing amount of HMGB1 or of active analogues or derivatives thereof, and diluents and/or emollients and/or adjuvants and/or excipients.

10. The medicated patch of claim 7, for topical use.

11. The medicated patch of claim 7, further comprising another pharmaceutically active compound.

12. The medicated patch according to claim 11 wherein the pharmacologically active compound is an anti-inflammatory compound.
